(19) United States
(12) Patent Application Publication
(10) Pub. No.: US 2013/0303946 A1
(43) Pub. Date: Nov. 14, 2013

(54) WEARABLE ARTICLE FOR DETECTING AN IMPACT AND METHOD OF OPERATION

(71) Applicant: WESTERN NEW ENGLAND UNIVERSITY, SPRINGFIELD, MA (US)

(72) Inventors: Robert T. T. Gettens, Longmeadow, MA (US); Nathan Kuzmeski, Tolland, CT (US); Lindsay Tencza, Middletown, CT (US); Nicholas St. John, Troy, NY (US); Thomas McLeod, Monson, MA (US); Philip Wahl, Canaan, NH (US); James C. McKeon, Longmeadow, MA (US); Glenn E. Vallee, Westbrook, CT (US)

(73) Assignee: WESTERN NEW ENGLAND UNIVERSITY, SPRINGFIELD, MA (US)

(21) Appl. No.: 13/888,441

(22) Filed: May 7, 2013

Related U.S. Application Data
(60) Provisional application No. 61/644,959, filed on May 9, 2012.

(51) Int. Cl. A61B 5/11

(52) U.S. Cl. CPC A61B 5/11
USPC 600/587

(57) ABSTRACT

A wearable article for detecting an impact to the head is provided. The wearable article includes a first flexible layer and a second flexible layer. A sensor is disposed between the first flexible layer and the second flexible layer. An indicator device is operably coupled to the sensor, the indicator device being configured to to provide a visual indication of the level of the impact.

### CROSS-REFERENCE TO RELATED APPLICATIONS

The present application claims priority to U.S. Provisional Application 61/644,959 filed on May 9, 2012 entitled “A Wearable Article for Detecting an Impact and Method of Operation”, the contents of which are incorporated by reference herein in their entirety.

### BACKGROUND OF THE INVENTION

The subject matter disclosed herein relates to a wearable article for sports and other activities and in particular to a wearable article that measures impacts to a user's head.

A concussion is a bruising of the brain caused by a blunt force blow that causes the brain to impact the skull. The blow may be directly to the head or may be the result of the head being hit by the inside of a helmet or headgear due to an impact force on the helmet. It should be appreciated that even though a helmet is intended to absorb impacts, a significant amount of force may still transfer through the helmet causing a concussion.

Athletes, at the professional and youth levels, suffer head injuries/concussions. Since signs, symptoms, and after effects of a concussion may be subtle, the injury may be undetected by coaches, trainer, parents and medical providers. The effect of multiple, even low level, concussions may result in long-term or permanent disability of the athlete. As a result, participants and the respective coaches, trainers, doctors, and parents have implemented additional procedures and diligence in the examination of athletes to assist in detecting the injury.

Many activities that cause concussion are recognized and participants wear protective helmets or headgear to absorb the impact of the blow. Athletes participating in some sports, such as football, hockey, lacrosse and other contact sports wear helmets to protect themselves. Laws have been passed that require children and adults who ride bikes, skate, skateboard, ski, snowboard or other activities to also wear helmets. In some games, such as soccer for example, the participants do not wear headgear. In still other sports, such as baseball for example, the players may only wear batting helmets during a portion of the game, such as while at bat. But during other portions of the game, such as in the field, they may wear non-protective hats. As a result, players may collide in play or hit the fence catching a long fly ball and suffer blows to the head that may cause concussions.

It should be appreciated that while existing methods of detecting concussions are suitable for their intended purposes the need for improvement remains, particularly in providing a wearable article that may measure impacts to the head to detect potential concussions.

### BRIEF DESCRIPTION OF THE INVENTION

A wearable article for detecting an impact to the head is provided. The wearable article includes a first flexible layer and a second flexible layer. A sensor is disposed between the first flexible layer and the second flexible layer. An indicator device is operably coupled to the sensor, the indicator device being configured to provide a visual indication of the level of the impact.

According to another aspect of the invention, a system for measuring an impact to a head is provided. The system including a wearable article having a first layer and a second layer. At least one sensor is disposed between the first layer and the second layer. A communications device is electrically coupled to the at least one sensor, the communications device being coupled to the first layer and the second layer. A receiver device is coupled for communication to the communications device.

According to yet another aspect of the invention, a method of detecting an impact to a head of a user is provided. The method includes providing a wearable article with a first layer and a second layer. At least one sensor is provided that is disposed between the first layer and the second layer. A communications device is provided that is coupled to at least one of the first layer and the second layer, the communications device being coupled to the at least one sensor. The wearable article is disposed on the head of the user. The impact is measured with the at least one sensor. A first signal is transmitted with the at least one sensor to the communications device in response to measuring the impact. A second signal is transmitted with the communications device.

These and other advantages and features will become more apparent from the following description taken in conjunction with the drawings.

### BRIEF DESCRIPTION OF THE DRAWING

The subject matter, which is regarded as the invention, is particularly pointed out and distinctly claimed in the claims at the conclusion of the specification. The foregoing and other features, and advantages of the invention are apparent from the following detailed description taken in conjunction with the accompanying drawings in which:

FIG. 1 is a perspective view of a wearable article in accordance with an embodiment of the invention;

FIG. 2 is a partial side sectional view of the wearable article of FIG. 1;

FIG. 3 is a side view of the wearable article of FIG. 1;

FIG. 4 is a side view of the wearable article of FIG. 1 in an opened position;

FIG. 5 is a perspective view of a wearable article in accordance with another embodiment of the invention;

FIG. 6 is a perspective view of a wearable article in accordance with another embodiment of the invention;

FIG. 7 is a side view of a wearable article in accordance with another embodiment of the invention;

FIG. 8 is a perspective view of a sensor for use with the wearable article of FIGS. 1 and 5-7;

FIG. 9 is a view of a monitoring device for use with the wearable article of FIGS. 1 and 5-7;

FIG. 10 is another view of the monitoring device of FIG. 9; and

FIG. 11 is a screen display illustration of data from the wearable article of FIGS. 1 and 5-7.

The detailed description explains embodiments of the invention, together with advantages and features, by way of example with reference to the drawings.

### DETAILED DESCRIPTION OF THE INVENTION

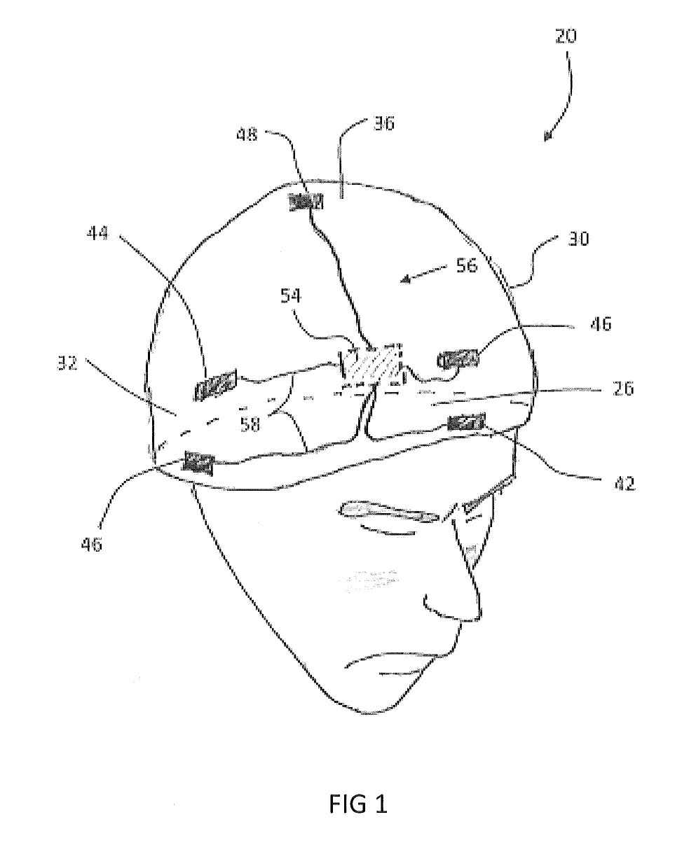

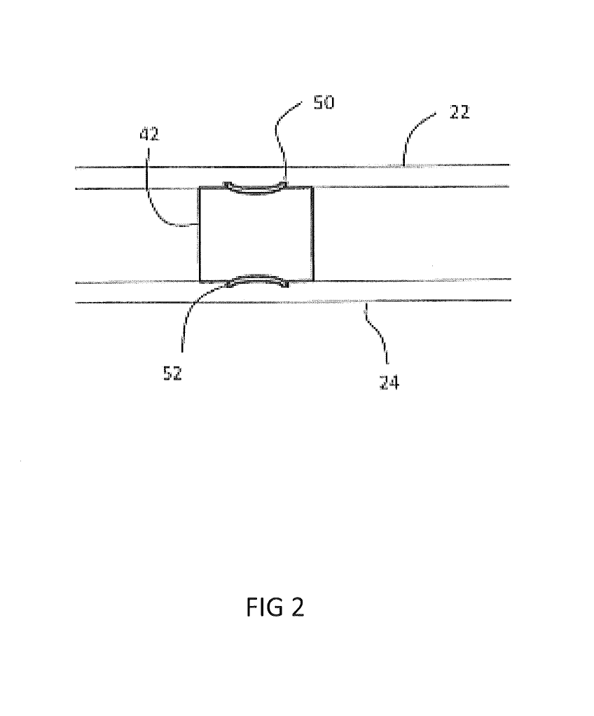

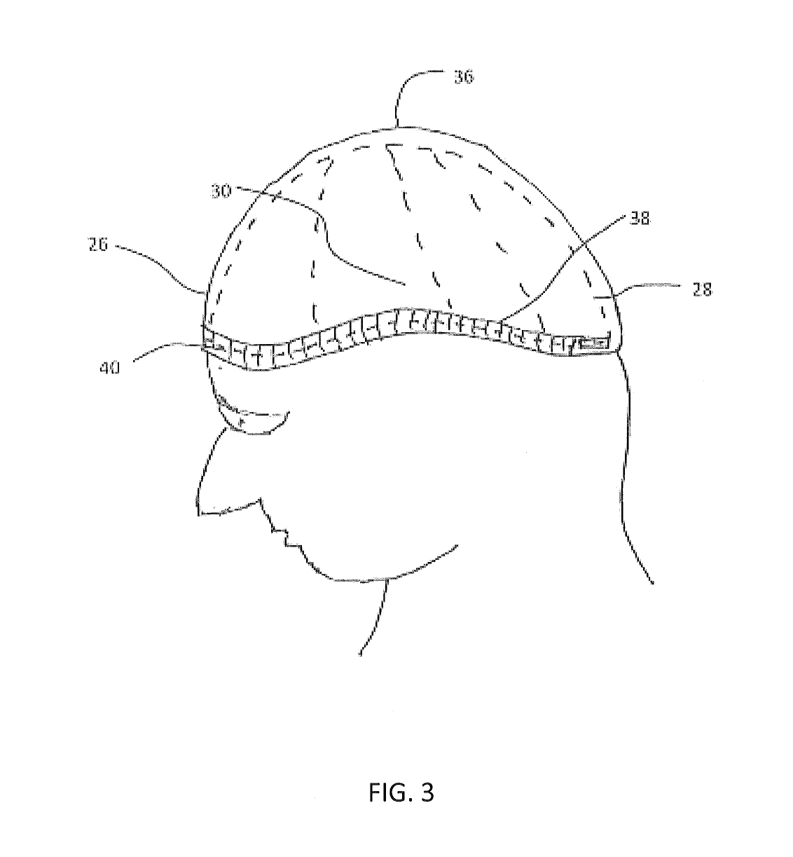

Concussions are an injury to the brain that may occur with hard to detect side effects. As a result, concussions may occur without the participant or others such as coaches or parents knowing that an injury has been sustained. When a person suffers additional subsequent concussions before an earlier concussion has healed, the person may suffer long term or permanent cognitive disabilities. Referring to FIGS. 1-4, a wearable article 20 is shown for measuring impacts to a person's head during an activity. Embodiments of this invention are configured to be worn as a stand-alone article, under head protection, such as a helmet for example, or under non-protective headgear, such as a baseball cap for example.

In the exemplary embodiment, the wearable article 20 includes a pair of adjacent layers 22, 24. The layers 22, 24 may be made from flexible materials that are shaped to fit tightly on a user's head. As will be discussed in more detail below, the layers 22, 24 form a shell for carrying sensors and transmitters that monitor for impacts. The layer 22 forms the outer layer of the shell and the layer 24 forms the inner layer of the shell. As used herein the term “inner” means closer to the users head, while the term “outer” means a position further from the users head. The layers 22, 24 may be made from a suitable elastic material, such as but not limited to nylon, cotton, latex, or a polyurethane-polyurea copolymer such as Lycra manufactured by Invista.

The layers 22, 24 are formed generally in a shape that conforms with the users head. The layers 22, 24 have a frontal portion 26, an opposing occipital portion 28 and a pair of opposing temporal portions 30, 32. A center portion 34 forms the area between the frontal portion 26, the occipital portion 28 and the temporal portions 30, 32. In the exemplary embodiment, the layers 22, 24 are fastened along a peripheral edge 38 by a fastening member, such as a zipper 40. The fastener 40 provides an advantage in allowing the wearable article 20 to be quickly disassembled by the user for cleaning and repair. The fastener 40 may also be, but is not limited to a hook-and-loop type fastener for example.

Disposed between the layers 22, 24 are a plurality of sensors 42, 44, 46, 48 that are arranged in the frontal 26, occipital 28, temporal 30, 32 and center 34 portions of the wearable article 20 respectively. In the exemplary embodiment, the sensors 42, 44, 46, 48 are fastened to the layers 22, 24 by one or more clips 50, 52 that allow the sensors 42, 44, 46, 48 to be removably coupled to the wearable article 20. In the exemplary embodiment, the sensor is a 3-axis accelerometer such as a MEMS motion sensor Model LIS302DL manufactured by STMicroelectronics for example. As will be discussed in more detail below, the sensors may also be, but are not limited to one dimensional accelerometers, strain gages, load cells, pressure transducers and the like.

The wearable article 20 further includes a communications device, such as a wireless transmitter 54. In the exemplary embodiment, the wireless transmitter 54 is compliant with a wireless protocol such as the Bluetooth protocol for example. The wireless transmitter 54 is electrically coupled for communication with each of the sensors 42, 44, 46, 48 by a circuit 56 that includes conductors 58. In one embodiment, the conductors 58 are covered with an epoxy or silicone layer that makes the circuit 56 water resistant. It should be appreciated that this provides advantages in reducing the potential for short circuits within the circuit 56 to moisture such as sweat from the user.

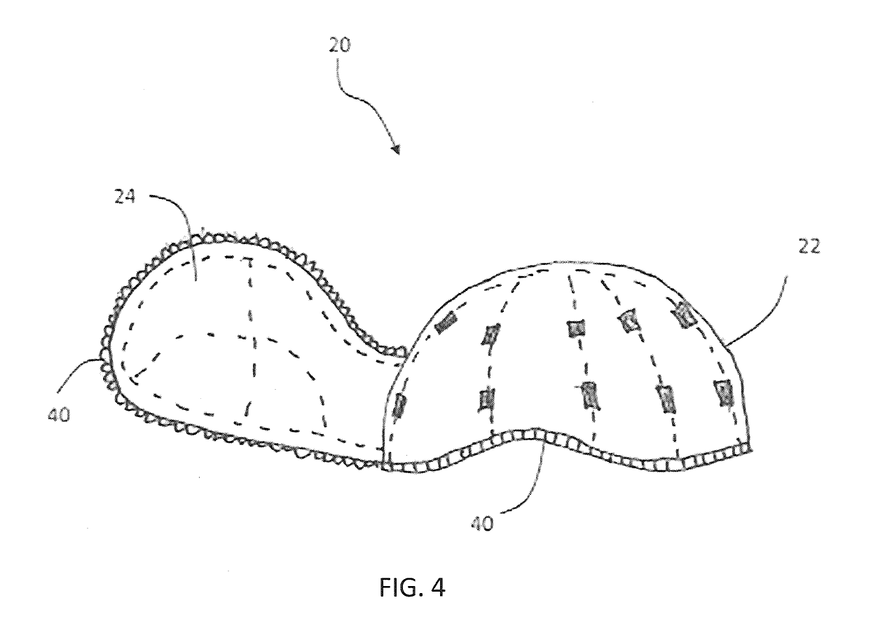

To access the sensors 42, 44, 46, 48 and the wireless transmitter 54, the user releases the fastener 40 along a substantial portion of the periphery. This allows the inner layer 24 to be moved away from the outer layer 22 as shown in FIG. 4 to provide access that allows removal of the internal components. It should be appreciated that this provides advantages in allowing the user to repair the wearable article 20 and for cleaning purposes.

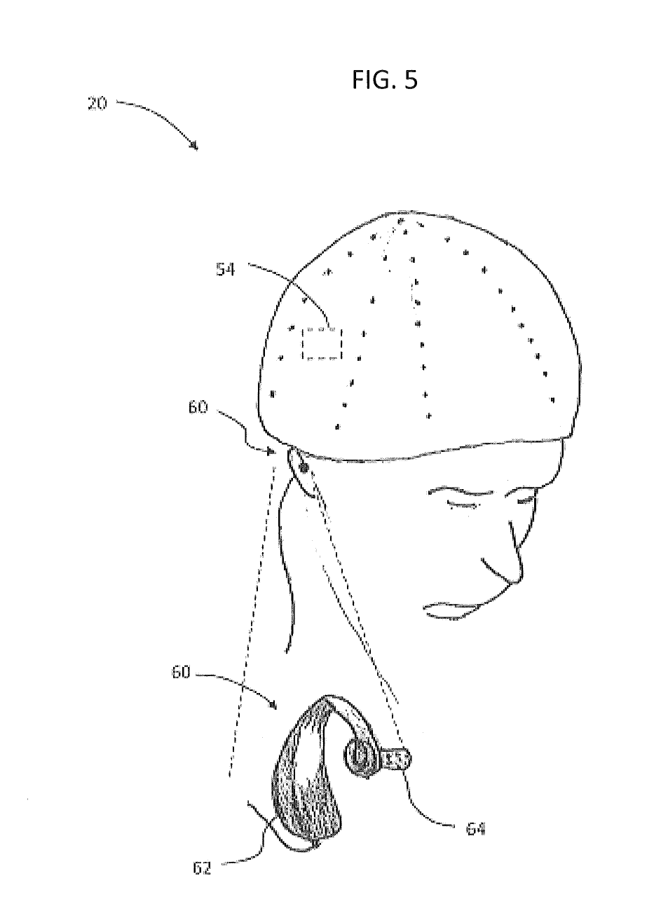

Another embodiment of the wearable article 20 is shown in FIG. 5. In this embodiment, communications device 54 is coupled to a hearing device 60. The hearing device 60 is sized and shaped to be coupled with the user's ear. In the exemplary embodiment, the hearing device 60 includes a body portion 62 that wraps around the back side of the user's ear. The hearing device 60 also includes a projection 64 on a distal end that extends into the user's ear. The projection 64 includes an audio device, such as a speaker for example, that allows an audible alarm to be emitted, notifying the user of a potential concussion. In this embodiment, the hearing device 60 or communications device 54 may have a processor (not shown) that interprets the measurement signals from the sensors 42, 44, 46, 48. The processor then causes an audible alarm to sound when a measurement signal, or a combination of measurement signals exceeds a predetermined threshold.

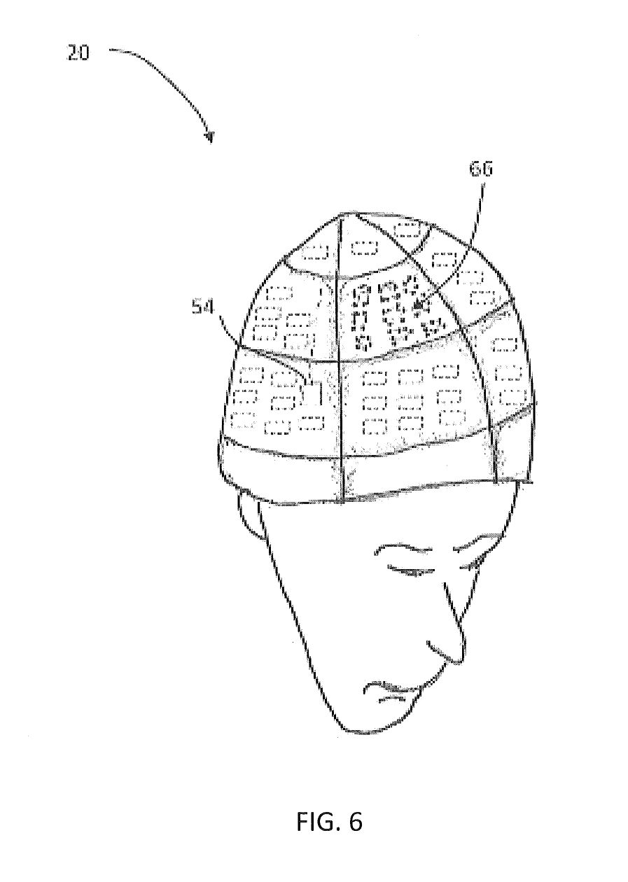

Yet another embodiment of the wearable article 20 is shown in FIG. 6. In this embodiment, the sensors 42, 44, 46, 48 are replaced with a plurality of pressure transducers, strain gauges, or load cells 66. The load cells 66 are distributed about the wearable article 20. The load cells 66 measure an input force or pressure directly. This embodiment provides advantages in that the close spacing of the load cells 66 allows a pressure or force map of the impacts to the user's head to be determined.

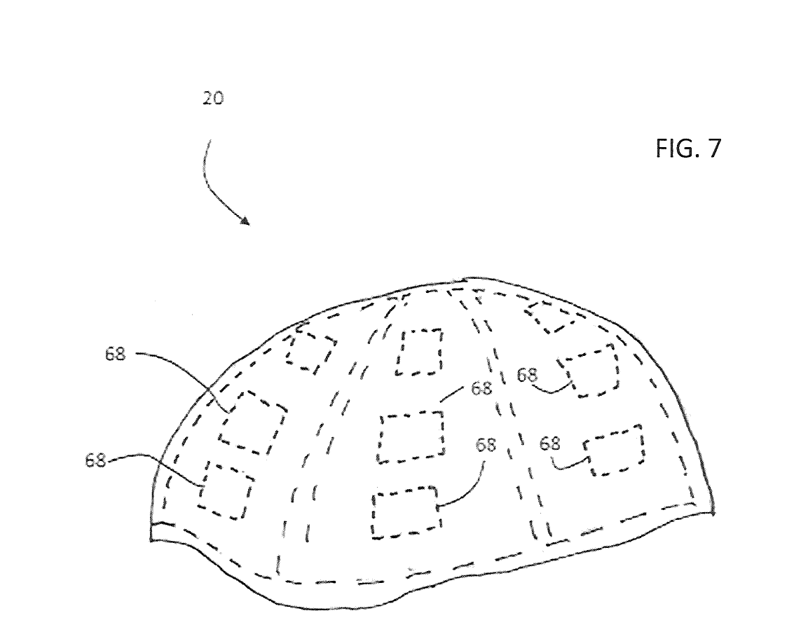

Still another embodiment of the wearable article 20 is shown in FIG. 7. In this embodiment, the wearable article 20 includes a plurality of pockets 68. Within each of the pockets 68 is disposed a replaceable color activation pouch. In this embodiment, the pouch changes color in response to a force or acceleration of a predetermined magnitude. In one embodiment, the pouch may contain a plurality of separate chambers that burst and mix upon impact. The color then provides a visual indication that an impact with significant magnitude has occurred and that the user may be suffering from a concussion.

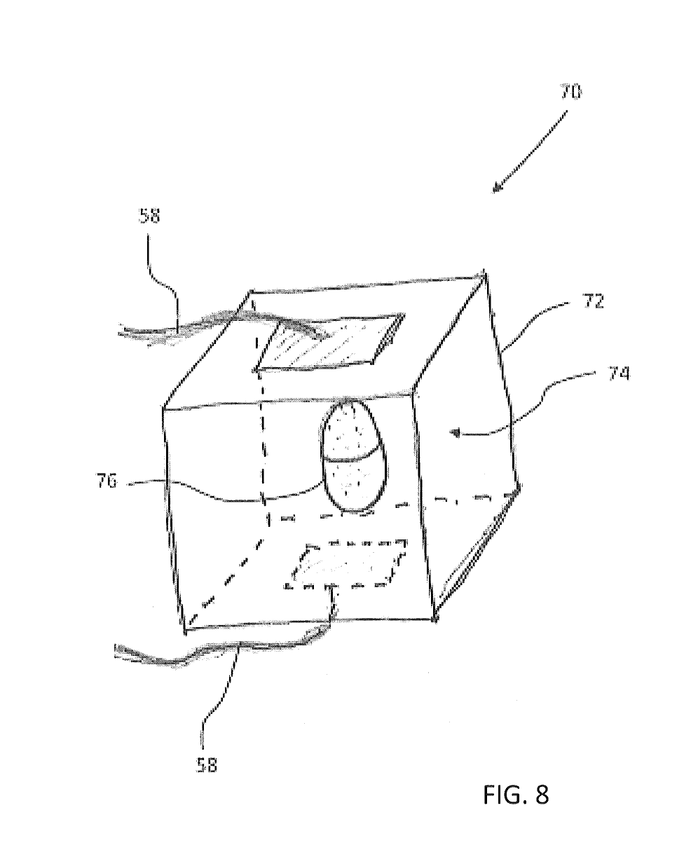

In one embodiment, each of the pockets 68 may include an impedance device 70, such as is shown in FIG. 8. In this embodiment, a device 70 is provided having a housing 72. On opposing sides 74, 76 of the housing 72, electrical conductors 58 are connected. Within the housing 72 is a solution 74 with a predetermined impedance. Suspended within the solution 74 is a breakable insert 76 containing a salt solution. The insert 76 is configured to break upon an impact of predetermined level. Once the insert 76 breaks, the salt solution mixes with the housing solution 74 causing the impedance between the conductors 58 to change. Thus, an impact capable of causing a concussion may be measured.

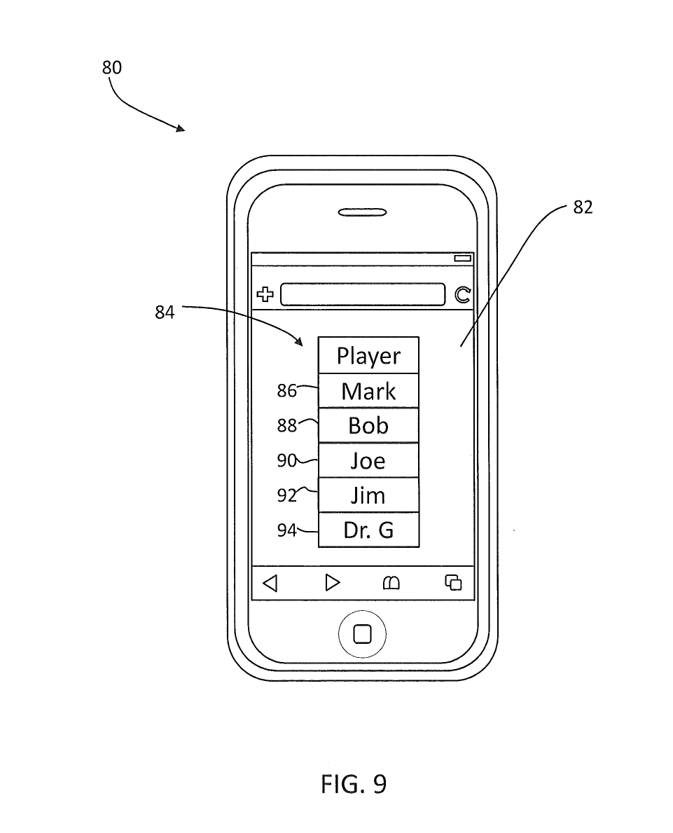

The wearable article 20 is paired via the communication device 54 to a receiver device 80. In one embodiment shown in FIG. 9, the receiver device 80 is a cellular phone or other portable electronic device that is configured to receive signals from the communications device 54. In the exemplary embodiment, the device 80 is configured to communicate with the wearable article 20 via a Bluetooth communications protocol. In other embodiments, the device 80 communicates with the wearable article via a communications system compliant with but not limited to: the IEEE 802.11 standard, the IEEE 802.15.4 standard (Zigbee, Ultra-wideband), and Wireless Universal Serial Bus standards (USB), ISO/IEC 18000-7 (DASH7) standard, and ISO/IEC 18000-3 (Near Field Communication) standard.

The receiver device 80 includes a screen 82 that provides a visual indicator 84 to the operator (e.g. coaching staff, trainer) of the impacts received by the players in an activity that are wearing wearable articles 20.

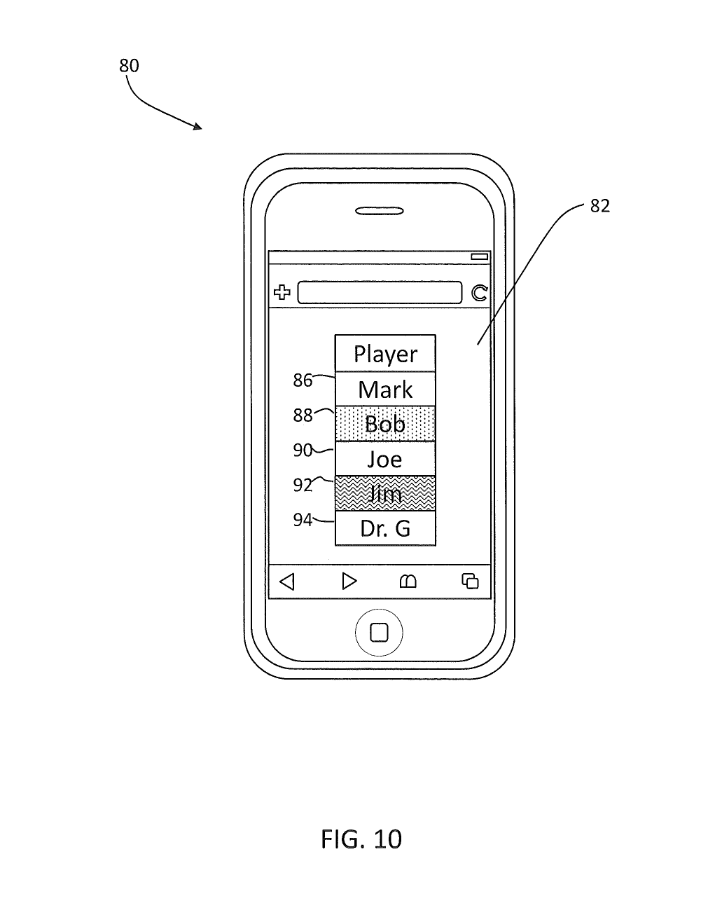

In one embodiment, the visual indicator 84 include an array of objects 86, 88, 90, 92, 94 that are each assigned to represent the impact status of a player having a wearable article 20. The objects may further include a textual element, such as a name or a jersey number for example, that allows the operator to identify the object with a particular player. The objects may further be displayed with color, such as red, yellow or green for example, where each color has an assigned meaning to the operator. For example, a green color (FIG. 10, objects 86, 92, 94) may indicate that the player has not received an impact that exceeds a threshold where a potential concussion would be of a concern, while a yellow color (FIG. 10, object 90) may indicate a potential concussion and a red color (FIG. 10, object 88) indicates a high risk of concussion.

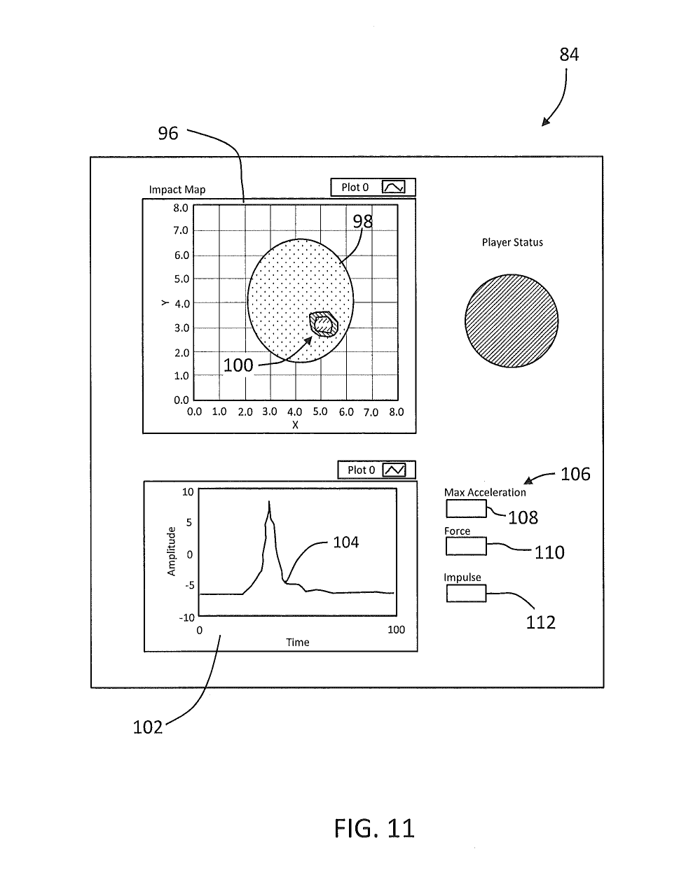

Referring now to FIG. 11, another embodiment of the visual indicator 84 is shown. In this embodiment, the indicator 84 includes a plurality of objects that displays additional data regarding data measured by a particular wearable article 20. The first object 96 includes a graphical representation 98 showing the outline of the wearable article 20. Superimposed on the outline 98 is a colored representation 100 of the location and level of impact on the wearable article 20. It should be appreciated that the colored representation 100 or map of the impact may be created by extrapolating data from the sensors of the wearable article 20 to represent the areas between the physical sensors.

The indicator 84 may further include a second object 102 showing a graphical representation 104 of the level or amplitude of the impact over time. Adjacent the second object 102 may be one or more textual objects 106 that display different parameters related to the impact, such as but not limited to the maximum acceleration 108, the force 110, and impulse 112. The indicator 84 may further include one or more graphical indicators, such as indicator 114 that display a different color depending on the level of impact as discussed above with respect to objects 86, 88, 90, 92, 94.

In one embodiment, the detailed information displayed on indicator 84 in FIG. 11 may be accessed by selecting one of the objects 86, 88, 90, 92, 94. In another embodiment, the indicator 84 of FIG. 11 may be created after the monitored activity, such as by medical personnel for example, to assist in diagnosing a potential concussion. In still another embodiment, the indicator 84 of FIG. 11 may be shown continuously. In this embodiment, the device 80 may periodically change the indicator to a different wearable article 20. In still yet another embodiment, the device 80 may have a sufficiently large screen, such as on a personal computer, a tablet computer or a laptop computer for example, to display multiple indicator 84 of FIG. 11 on one screen to allow the operators to monitor the impacts on multiple players simultaneously.

In operation, the wearable article 20 is coupled for communication to the device 80. During use the wearable article 20 transmits data continuously, periodically or on an aperiodic basis to the device 80. The device 80 receives the signal from the wireless transmitter 54. When one of the sensors 42, 44, 46, 48 measures an impact on the wearable article 20, a signal is transmitted to the wireless transmitter 54 which, in turn, transmits a signal to the device 80. In one embodiment, the wireless transmitter 54 may include a processor and memory that allows data from the sensors 42, 44, 46, 48 to be buffered prior to being transmitted to the device 80. In another embodiment, the wireless transmitter 54 may perform calculations on the signal received from the sensors 42, 44, 46, 48, such as to accommodate calibration parameters for example, prior to transmitting the signal to the device 80.

The device 80, upon receiving the signal from the wearable article 20, compares the level of impact measured by the sensors 42, 44, 46, 48 against a threshold. If the impact level exceeds a first threshold, the device 80 changes the color of the object 86, 88, 90, 92, 94 that is associated with that data to indicate that a potential concussion level impact was measured (e.g. color yellow). If the impact level exceeds a second threshold that indicates a high risk of concussion, the device 80 changes the color associated with the object 86, 88, 90, 92, 94 that is associated with that data (e.g. color red). In the case of a high risk impact, the operator of the device 80 may then halt the activity to check the on the wearer of the wearable article 20.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

1. A wearable article for detecting an impact to the head:
a first flexible layer;
a second flexible layer;
a sensor removably coupled between the first flexible layer and the second flexible layer; and
an indicator device operably coupled to the sensor, the indicator device being configured to provide a visual indication of a level of the impact.

2. The wearable article of claim 1 further comprising a communications device electrically coupled to the sensor and disposed between the first flexible layer and the second flexible layer, the communications device being coupled for communication with the indicator device.

3. The article of claim 2 wherein the first flexible layer has a first occipital portion and the second flexible layer has a second occipital portion, the communications device being disposed between the first occipital portion and the second occipital portion.

4. The article of claim 3 where the first flexible layer and the second flexible layer include a pair of opposing temporal portions, the sensor being disposed in one of the temporal portions.

5. The article of claim 4 wherein the sensor includes a first sensor and a second sensor, the first sensor being position in one of the temporal portions and the second sensor being disposed in the other of the temporal portions.

6. The article of claim 1 wherein the sensor is an accelerometer or a load cell.

7. The article of claim 2 further comprising a conductor electrically coupling the sensor to the communications device, the conductor including a layer resistant to water.

8. A system for measuring an impact to a head, the system comprising:
a wearable article having a first layer and a second layer, at least one sensor disposed between the first layer and the second layer, and a communications device electrically coupled to the at least one sensor, the communications device being coupled to the first layer and the second layer; and
a receiver device coupled for communication to the communications device.

9. The system of claim 8 wherein the receiver device includes a display.

10. The system of claim 9 wherein the receiver device includes a processor responsive to executable instructions when executed on the processor for displaying an indication of the display of a location and level of the impact in response to a signal from the at least one sensor.

11. The system of claim 9 wherein the receiver device includes an audio device, the audio device configured to emit an alarm in response to a signal from the at least one sensor.

12. The system of claim 9 wherein the at least one sensor includes a first sensor disposed in the first portion, a second sensor disposed in the second portion and a third sensor in a third portion opposite the second portion.

13. The system of claim 12 wherein the first sensor, the second sensor and the third sensor are accelerometers or load cells.

14. The system of claim 10 wherein the receiver device includes a display, the display being configured to display a plurality of objects, the color of the objects being based at least in part on the signal from the at least one sensor.

15. The system of claim 10 wherein the receiver device includes a display, the display configured to display an indicator object and a graphical representation of the wearable article.

16. A method of detecting an impact to a head of a user, the method comprising:
providing a wearable article with a first layer and a second layer;
providing at least one sensor disposed between the first layer and the second layer;
providing a communications device coupled to at least one of the first layer and the second layer, the communications device being coupled to the at least one sensor;
disposing the wearable article on the head of the user;
measuring the impact with the at least one sensor;
transmitting a first signal with the at least one sensor to the communications device in response to measuring the impact; and
transmitting a second signal with the communications device.

17. The method of claim 16 further comprising:
providing a receiving device having a signal device coupled for communication to the communications device;
receiving the second signal with the signal device; and
emitting an alarm in response to the signal device receiving the second signal.

18. The method of claim 17 further comprising measuring an acceleration with the at least one sensor.

19. The method of claim 17 further comprising measuring a force with the at least one sensor.

20. The method of claim 17 further comprising transmitting said second signal from the communications device to the signal device wirelessly.
